(19) United States
(12) Patent Application Publication
(10) Pub. No.: US 2025/0000231 A1
(43) Pub. Date: Jan. 2, 2025

(54) ARTIFICIAL NAIL FOR USE IN THE TREATMENT OF A NAIL DISEASE

(71) Applicant: ONICOR B.V., Tilburg (NL)

(72) Inventors: Ronald Van Der Geest, Tilburg (NL); Oscar VAN LOOSBROEK, Tilburg (NL)

(73) Assignee: ONICOR B.V., Tilburg (NL)

(21) Appl. No.: 18/292,174

(22) PCT Filed: Aug. 4, 2022
(86) PCT No.: PCT/EP2022/072016

§ 371 Date: Jan. 25, 2024

(30) Foreign Application Priority Data
Aug. 5, 2021 (NL) 2028927

(51) Int. Cl. A45D 31/00; A45D 34/04

(52) U.S. Cl. CPC A45D 31/00; A45D 34/04

(57) ABSTRACT

An artificial nail for use in the treatment of a nail disease, in particular a fungal disease of the nail. Methods for treating a nail disease using the artificial nail for prolonged and effective exposure of the antifungal composition on the infected nail, including a cosmetic treatment of the infected nail.

The present invention relates to an artificial nail for use in the treatment of a nail disease, in particular a fungal disease of the nail. The present invention further relates to methods for treating a nail disease using said artificial nail for prolonged and effective exposure of the antifungal composition on the infected nail, including a cosmetic treatment of the infected nail.

Onychomycosis is a fungal infection of the nail and is the most common disease of the nails and constitutes about half of all nail abnormalities. It occurs in about 10% of the adult population. Onychomycosis can affect both toenails and fingernails, but toenail infections are particularly common. One of the most common symptoms of a fungal nail infection is the thickening and discoloring (mostly white or yellow) of the nails. As the infection progresses, the nail becomes brittle, with pieces breaking off from the toenail or fingernail. If left untreated, the skin can become inflamed and painful underneath and around the nail. From a cosmetic point of view, nail infections can be unpleasant sight and may cause significant bodily discomfort.

Several methods for treating nail infection are available, including systemic treatments (tablets or capsules) and topical treatments (e.g. crèmes and lacquers). The systemic treatment approach consists of administering a subject suffering from a nail infection with an antimicrobial or antifungal compound or drug, which are well-absorbed from the gastro-intestinal tract. Such compounds are formulated as tablets or capsules and dosed continuously or with timed intervals to patients for treatment of the nail infection. Topical treatments are applied directly onto the nails.

However, fungal nail infections are known to accumulate in the deeper layers of the skin and nailbed. These regions are not vascularized and therefore poorly or not reached at all by systemic treatment, e.g. via the systemic circulation. This in turn causes the outgrowth of healthy nails while the fungal infection continues to grow into the deeper parts of the nail. Another limitation associated with the systemic treatment approaches is the need for prolonged treatment periods (e.g. at least about three to six months) due to the slow growth of the nails which in turn leads to the occurrence of potential systemic drug interactions and side effects, such as systemic toxicities, drug-drug interactions and in some case even severe liver toxicity. Taken together, these limitations contribute to limited compliance with the treatment (e.g. subjects is likely to discontinue treatment) and ensue that poor cure rates are observed.

Topical treatments are applied directly onto the nails. The topical treatment approach consists of applying the active compound or drug (e.g. in the form of a lacquer or crème) having anti-fungal or antimicrobial activity directly onto the surface of the infected nail. However, since the nail is an excellent natural rigid barrier against the ingress of foreign material, it also prevents the drug component to reach the infected site of the nail to be effective. Therefore, the use of topical drugs to directly treat the nail has been rather ineffective, because of its poor penetration across the layers of the nail. Other topical treatment approaches aimed at softening the nail surface (e.g. targeting α-keratin) to improve penetration of therapeutic compounds have been attempted but have also been found to be marginally successful. Furthermore, topical treatments are typically left to dry after application. As a result of this procedure, drug substance and any additives that constitute the formulation will precipitate in the higher regions (superficial layers or parts) of the nail without much access or penetration into the nail bed. Precipitated drug will not be active and in fact will clog up any channels that would have allowed access by diffusion to the deeper regions of the nail, if it would have remained solubilized.

Therefore, a main problem associated with the treatment of onychomycosis is that currently existing systemic treatments (tablets or capsules) and topical treatments applied onto the nails have very poor efficacy and often fail to cure the condition (nail disease, e.g. onychomycosis). Strategies to treat onychomycosis topically are constrained by the requirements of the formulation. Lacquers have to harden within a specific amount of time. Crèmes need the right viscosity, since an increase in fluidity of the antifungal composition improves nail penetration, however it also affects the evaporation and time of exposure of the active compound at the infected site. Furthermore, a prolonged and effective exposure of a liquid antifungal composition at the site of treatment resulting in an effective treatment is very difficult to achieve. And in both cases, lacquer or crème, any excipient necessary to inhibit the fungal needs to be absorbed, get to the right location and bind their target before they crystallize, evaporate or get displaced.

Considering the above, there is a need in the art for a treatment of nail disease including a cosmetic treatment of said nail disease, such as onychomycosis, that has improved efficacy, such as improved penetration of the active compound into the nail or site of infection, is easy to use for a prolonged period of time to ensure compliance with the treatment, prevent side effects of exposure to a prolonged drug regiment, and ensure a high chance of successful treatment of the nail disease. In addition, there is a need for a treatment that alleviates the negative cosmetic adverse effects and body discomfort associated with nail infections.

It is an object of the present invention, amongst other objects, to address the above need in the art. The object of present invention, amongst other objects, is met by the present invention as outlined in the appended claims.

Specifically, the above object, amongst other objects, is met, according to a first aspect, by the present invention by an artificial nail for use in the treatment of a nail disease, wherein the artificial nail is in the shape of a nail for sealing of a nail and/or nail bed surface of a nail, wherein the artificial nail is comprised of a polymer surface side resembling the keratinous plate of a nail and a ventral side directed to a nail and/or nail bed to be treated, wherein said ventral side comprises one or more reservoirs for holding a liquid composition comprising an antimicrobial compound and/or wherein said ventral side comprises one or more protrusions for providing adhesion of said liquid composition to the ventral side of the polymer layer, and wherein said one or more reservoirs is configured to allow application of said liquid composition comprising an antimicrobial compound to said nail and/or nail bed to be treated.

The artificial nail of present invention provides a continued administration of the active antimicrobial compound(s) or therapeutic agents i.e. improved transungual delivery of the active compound to the site of infection or disease over an extended period of time. As a result, higher compliance (e.g. adherence) to treatment and significantly improved efficacy and/or cure rate is achieved. The one or more reservoirs are on the inner surface of the ventral side facing the surface area to be treated of the nail or nail bed of a finger or toe where the antimicrobial compound needs to be applied to for treatment. Prolonged exposure of the area to be treated of the nail with an antimicrobial compound in combination with the nail-specific sealing (i.e. occlusion that prevents evaporation of the liquid solution comprising the antimicrobial compound) allow sufficient time for the antimicrobial compound to reach the site of action in sufficient quantities in order to be effective, thus improved transungual delivery of the active compound to the site of infection or disease. The sealed connection between the artificial nail and the nail or nail bed to be treated is such that it is protected or shielded from the external environment (e.g. no or no substantial contact with ambient air). Under sealing or occlusion conditions, the nail has a better ability to absorb the liquid composition as well as the solubilized drugs therein, which means that the liquids comprising the solubilized drugs can readily diffuse to the deeper layers of the nail. A further advantage associated with the use of the artificial nail is that by occlusive covering of the infected nail, cross contamination by the infected nail or re-infection of the curing nail is prevented or reduced. The artificial will follow the contours of the visible nail plate from the inside accurately, whereby at least 90%, preferably at least 95%, more preferable at least 98%, even more preferable 99%, most preferable 100% of the nail plate surface is enclosed.

Furthermore, it is preferred that the artificial of present invention follows the contours of the visible nail plate from the outside accurately, to improve the therapeutic and cosmetic effect of the artificial nail Therefore the outer surface of the artificial nail of the invention may be comprised of a convex surface having a natural nail shape, while the inner surface, facing the infected nail, shall be composed of convex and concave zones, depending on the 3D geometry of the infected nail. The artificial nail is suitable to be applied onto a nail and/or nail bed by attachment to the edge of the cuticle, lateral nail folds and epidermis below the nail plate, preferably at a distal end of the nail plate having a maximum distance to the distal end of the nail plate of at most 1 mm. The distance between the artificial nail to the lateral nail folds and cuticle of said nail and/or nail bed to be treated is at most at most 1 mm.

The artificial nail of present invention comprises a ventral side that is comprised of one or more reservoirs for holding a liquid composition comprising an antimicrobial compound and wherein the ventral side comprises one or more protrusions for providing adhesion of said liquid composition to said ventral side. The one or more protrusions will reduce the gap the liquid meniscus needs to bridge and provide additional surface for the liquid to “cling to” on the ventral side of the artificial nail. As such, the protrusions result in a more even liquid film and improve the adhesion of the liquid to the artificial nail and providing an improved antimicrobial effect and treatment of the infected nail bed. Therefore, the liquid composition is held into place and is more dispersed on the second polymer when placed on the nail or nail bed to be affected and does not drip off that easily anymore.

Another advantage is that the artificial nail of present invention comprises an outer surface, the surface side that resembles a natural non-infected nail surface, i.e. the natural aspects of an uninfected nail, such as shape, colour, structure, size, translucency, thereby alleviating the unpleasant sight and body discomfort associated with nail disease by improving the visual or cosmetic aspects and further improving treatment compliance and thus additionally providing a cosmetic treatment of the infected nail. The artificial nail that is adapted to the particular natural nail to be treated, for example by using 3D printing technology.

The artificial nail of present invention does not have the constraints of time of application or incubation, viscosity or evaporation limitations of the antimicrobial composition, displacement issues, or issues with absorption of the compound at the area to be treated on the nail. The formulation can vary in liquidity, viscosity, it could be a solution, suspension, a gel, a liposomal formulation or a combination thereof, but preferably an aqueous, alcohol or solvent based liquid composition to improve impregnation of the infected nail or nail bed and improve treatment efficacy.

According to a preferred embodiment, the present invention relates to the artificial nail, wherein said one or more reservoirs is comprised of a liquid permeable polymer matrix for enabling flow through of the liquid composition and contact between the liquid composition and nail and/or nail bed to be treated, wherein the liquid permeable polymer matrix is comprised of one or more materials selected from the group consisting of keratin, polyester, polyurethane, polytetra-fluoroethylene, polyethylene, polysysloxane, polyisocyanate, poly methylmethacrylate, modified polyacrylonitrile, polyether imide, polylactide, polylglycolide, polycaprolactone, polyhyaluronic acid, cyclic olefin copolymer, collagen, chitosan cellulose acetate, and combinations thereof, preferably keratin and combinations thereof. The permeable polymer matrix may also comprise small apertures to further improve the distribution of the liquid composition in the artificial nail within the one or more reservoirs. The liquid permeable polymer matrix can be a liquid permeable polymer closure element, aperture and/or an end cap having an open sponge like structure trough which the liquid composition comprising the antimicrobial agent can flow and reach and come into direct and continues contact with the infected nail or nail bed during the duration of the treatment when the nail is positioned on the infected nail or nail bed for improved transungual delivery of the active compound to the site of infection or disease and efficacy of the treatment.

According to yet another preferred embodiment, the present invention relates to the artificial nail wherein said one or more protrusions have a 3D shape selected from the group consisting of mushroom, tree (i.e. branching columns or microvilli), gyroid, lattice, cubical, cone, cylinder, pyramid, and sphere, preferably mushroom shape. The specific 3D shape of the one or more protrusions provides an improved adhesive contact area for the liquid composition comprising an antimicrobial compound from the one or more reservoirs that is held between the nail to be treated and the second polymer nail of the artificial nail of present invention. These 3D structures improve the distribution of the liquid composition in the artificial nail and prevent drip off and spillage of the liquid composition during application of the artificial nail to the site to be treated. The liquid composition containing antimicrobial compounds may be in direct contact with the nail plate.

According to another preferred embodiment, the present invention relates to the artificial nail, wherein the polymer layer is comprised of one or more materials selected from the group consisting of keratin, polymethylmethacrylate, polyacrylate, polycarbonate, polyether, polyethersulfone, polyethylene terephthalate, polyisocyanate, polypropylene, polysiloxane, polystyrene, polysulfone, polyvinylchloride, polyvinyl butyral, polyurethane, cellulose acetate, chitosan, epoxie, preferably keratin and combinations thereof.

According to another preferred embodiment, the present invention relates to the artificial nail, wherein said one or more reservoirs is comprised of a liquid permeable polymer matrix that is further comprised of an adhesive polymer material selected from the group consisting of resin, rosin, TSF resin, polyepoxides, copal, latex, gum resin, styrax liquid, methyl salicylate, methacrylates, acrylates, diacrylates, triacrylates, sobornyl acrylate, dimethylacrylamid, ethyl cyanoacrylate, methacrylic acid, methyl methacrylate, N-butyl methacrylate, iso-butyl methacrylate, ethyl methacrylate, 2-ethylhexyl methacrylate, N,N-dimethyl-p-toluidine, 2-(2-hydroxy-5-methylphenyl) benzotriazole, 2-hydroxy-4-(octyloxy) benzophenone, (2H-benzotriazol-2-Yl)-4,6-ditertpentylphenol, polyfunctional amines, preferably isophorone diamine, and combinations thereof. The adhesive polymer material provides for improved adhesion of the polymer artificial nail tot the infected nail or nail bed and provides a further enclosure of the one or more reservoirs. The adhesive polymer material needs to be of liquid permeable polymer material to enable flow trough and continues contact of the liquid composition with the infected nail or nail bed surface. Furthermore, the artificial nail, when applied to on the infected nail may be further covered by an adhesive top layer, for example Tegaderm®.

According to another preferred embodiment, the present invention relates to the artificial nail, wherein the one or more reservoirs comprise a volume of 5 to 250 μl, preferably 10 to 150 μl, more preferably 25 to 125 μl, even more preferably 50 to 100 μl, most preferably 75 to 85 μl. A minimal volume in required as it will be easier to mimic the natural nail, depending on the effectivity of the liquid antimicrobial composition and the drug release rate is from the artificial nail.

According to yet another preferred embodiment, the present invention relates to the artificial nail, wherein the one or more reservoirs are at least two, preferably at least three, more preferably at least four, most preferably at least five reservoirs. The presence of multiple separate reservoirs enables a combination treatment of the nail disease wherein the reservoirs are filled with different liquid compositions comprising various antimicrobial compounds or different regimens. Another preferred embodiment would be a sequential administration of a different antimicrobial composition per week, for example week one a water-based composition, and week two an alcohol based composition comprising an antifungal. The liquid composition containing antimicrobial compounds is preferably in direct contact with the nail plate. Direct contact with the nail plate improves permeation and penetration of the drug into and through the nail plate.

According to another preferred embodiment, the present invention relates to the artificial nail, wherein the artificial nail comprises a rigid structure of non-uniform thickness, wherein the dorsal side of said nail has a smooth surface whereas the ventral side will follow the nail and/or nail bed to be treated, i.e. follow the altered anatomy of a nail plate affected by onychomycosis.

According to yet another preferred embodiment, the present invention relates to the artificial nail, wherein the artificial nail surface at the ventral side (3) is comprised of multiple convex and concave surfaces following the shape of the surface of the nail and/or nail bed to be treated (1) and/or following the contours of the nail and/or nail bed to be treated (1). Therefore the outer surface of the artificial nail of the invention shall may be comprised consist of a convex surface having a natural nail shape, while the inner surface, facing the infected nail, shall be composed of convex and concave zones, depending on the 3D geometry of the infected nail. Due to its exact shape, fit and occlusion characteristics, the artificial nail of present invention is preferably obtained via 3D printing. The process of 3D scanning and 3D printing enables to obtain the artificial nail having the shape and precision. The anatomy of affected nails is altered in Onychomycosis. Onychomycosis causes brittleness, causing parts to fall of, hyperkeratosis leading to nail thickening, and increases porosity and structural integrity of the nail plate leading to rougher surface. If there is no open compartment required, but direct contact with the nail plate (for example at the sides near the lateral folds) strong adhesion can be obtained without the need of abrasion of the nail plate by matching the geometric shapes of the surface in the 3D printed artificial nail.

The artificial nail is attached to the lateral nail folds, cuticle and epidermis at the distal end of the nail plate or in another embodiment the artificial nail is attached directly to the nail plate. The artificial nail of present invention can be attached to surrounding epidermis and enables and seals the entire/or full upper external facing part of the nail plate thereby improving hydration state. The lateral nail folds and epidermis at the distal end of the nail are places where liquid evaporates, affecting the hydration state of the nail plate. Since the hydration state and water content are thought to be one of the most important parameters affecting drug diffusion kinetics and drug permeation and penetration this is a feature that is very difficult to achieve and is preferably therefore replicated by using 3D printing in the production of the artificial nail of present invention.

According to yet another preferred embodiment, the present invention relates to the artificial nail, wherein the polymer layer is comprised of at least one UV-curable polymer. UV-curable polymer is a specific polymer group that can be readily 3D printed.

According to another preferred embodiment, the present invention relates to the artificial nail, wherein the polymer layer of the artificial nail comprises at least one valve or at least one aperture in combination with a closing means, such as a plug, for (re)filling of the one or more reservoirs (4) for holding a liquid composition comprising an antimicrobial compound. The valve may be a slit or crack within the flexible polymer layer, small enough to contain the liquid composition and maintain the sealed functionality of the artificial nail. However, the valve can be opened by an applied force, for example using a needle or syringe on the valve for re(filling) the reservoir of the artificial nail with the liquid composition.

The present invention, according to a second aspect, relates to a method for treating a nail disease using an artificial nail, comprising the step of
a) providing an artificial nail of present invention,
b) preparing the artificial nail by adding a liquid composition comprising an antimicrobial compound to the one or more reservoirs,
c) applying the prepared artificial nail onto a nail and/or nail bed to be treated thereby sealing off the nail and/or its nail bed from the environment.
   Preferably the method of present invention relates to a cosmetic method of treating a nail disease using the artificial nail as disclosed above. The method provides on top of the infected nail the artificial nail of present invention having an outer surface, the surface side that resembles a natural non-infected nail surface, i.e. the natural aspects of an uninfected nail, such as shape, colour, structure, size, thereby alleviating the unpleasant sight and body discomfort associated with nail disease by improving the visual or cosmetic aspects and further improving treatment compliance and providing a cosmetic treatment of the infected nail. The artificial nail may comprise means for adhesion to the natural nail to be treated. Similarly, the artificial nail may be attached on the natural nail to be treated with an additional adhesive cover layer. The sealed connection between the artificial nail and the nail or nail bed to be treated is such that it is protected or shielded from the external environment (e.g. no or no substantial contact with ambient air).

According to another preferred embodiment, the present invention relates to the method for treating a nail disease using an artificial nail, wherein applying the prepared artificial nail comprises that the one or more reservoirs are in direct contact with the nail and/or its nail bed to be treated.

According to yet another preferred embodiment, the present invention relates to the method for treating a nail disease using an artificial nail, wherein the liquid composition comprising an antimicrobial compound is a solution comprising water and/or alcohol, propylene glycol, hydroxypropyl-b-cyclodextyrin, hydrochloric acid, sodium hydroxide, and an antimicrobial compound. A water or alcohol based liquid compositions facilitate drug transport across the nail by enhancing nail swelling which results in greater drug mobility in the barrier and pore formation in the infected nail or nail bed. The liquid composition may be any fluid, a gel, suspension, or colloidal system (such as comprising liposomes, or micelles). Water based compositions increase the nail permeability.

According to another preferred embodiment, the present invention relates to the method for treating a nail disease using an artificial nail, wherein the liquid composition further comprises one or more compounds comprising a sulfhydryl (—SH) group selected from the group consisting of acetylcysteine, cysteine, mercaptoethanol, and thioglycolic acid, preferably acetylcysteine. Compounds which contain sulfhydryl (—SH) groups such as acetylcysteine, cysteine, mercaptoethanol, can reduce, thus cleave the disulphide bonds in nail proteins and destabilises the keratin network of the nail, which leads to increased permeability for the antimicrobial agent.

According to a preferred embodiment, the present invention relates to the method for treating a nail disease using an artificial nail, wherein abrasion of said nail and/or nail bed to be treated is performed before application of the prepared artificial nail onto to a nail and/or nail bed to be treated. Abrasion may help in the case of thickened nails to allow proper attachment of the artificial nail to the underlying nail and/or residual structure. The nail is an excellent barrier against the ingress of foreign material, but as a consequence it also prevents effective topical treatment. The nail is composed of α-keratin, which provides structural rigidity. Mechanical methods like abrasion of the nail can be effective to further decrease the structural integrity of the nail to be treated by partial removal of the nail plate resulting in increased penetration of the antimicrobial compound at the site of infection or disease.

According to another preferred embodiment, the present invention relates to the method for treating a nail disease using an artificial nail, wherein the antimicrobial compound is one or more selected from the group consisting of antimycotic agent, itraconazole, tavaborole, efinaconazole, terbinafine, miconazole, clotrimazole, bifonazole, butoconazole, econazole, fenticonazole, ketoconazole, oxiconazole, sulconazole, voriconazole, albaconazole, fluconazole, ravuconazole, amorolfine, butenafine, chlorhexidine, naftifine, andilafungin, caspofungin, micafungin, benzoic acid, ciclopirox, tolnaftate, undecylenic acid, crystal violet, methylene blue, preferably itraconazole.

According to yet another preferred embodiment, the present invention relates to the method for treating a nail disease using an artificial nail, wherein the nail disease is one or more selected from the group consisting of onychomycosis, pseudomonas infection, psoriasis of the nail, and paronychia, preferably onychomycosis.

According to a preferred embodiment, the present invention relates to the method for treating a nail disease using an artificial nail, wherein the prepared artificial nail is kept onto said nail and/or nail bed to be treated for a period of at least 1 day, preferably 1 week, more preferably at least two weeks, even more preferably at least 4 weeks, most preferably at least 2 months.

According to a preferred embodiment, the present invention relates to the method for treating a nail disease using an artificial nail, wherein the artificial nail is applied onto a nail and/or nail bed by attachment to the edge of the cuticle, lateral nail folds and epidermis below the nail plate, preferably at a distal end of the nail plate having a maximum distance to the distal end of the nail plate of at most 1 mm, preferably at most 0.5 mm, more preferably at most 0.2 mm, even more preferably at most 0.1 mm, preferably at most 0.05 mm, most preferably at most 0.01 mm. When the artificial nail of the present invention is attached to nail plate, the further away from the distal end of the nail plate, the more surface is not covered by the antimicrobial formulation and consequently be exposed to lower concentrations of antifungals. Additionally, exposed surface nail plate will be exposed to air and may result in a decreased hydration state.

According to another preferred embodiment, the present invention relates to the method for treating a nail disease using an artificial nail, wherein the distance between the artificial nail to the lateral nail folds and cuticle of said nail and/or nail bed to be treated is at most at most 1 mm, preferably at most 0.5 mm, more preferably at most 0.2 mm, even more preferably at most 0.1 mm, preferably at most 0.05 mm, most preferably at most 0.01 mm. The lateral nail folds and epidermis at the distal end of the nail are places where liquid evaporates, affecting the hydration state of the nail plate. Therefore, positioning of the artificial nail as indicated above prevents or reduces the evaporation effect, thereby improving the antimicrobial efficacy since the hydration state and water content are thought to be one of the most important parameters affecting drug diffusion kinetics. If artificial nail is attached to surrounding epidermis (nail cuticles) further distanced from nail plate is achieved leading to a less natural look and feel of the artificial nail, resulting in decreased wearing comfort and exposes surround healthy epidermis to drug unnecessarily. If artificial nail is attached to nail plate only, it only seals in nail plate surface, which might be preferred if lateral nail fold and nail cuticle exposure to air is required for healthy homeostasis of the nail plate environment.

The present invention, according to a further aspect, relates to the use of an artificial nail of present invention for the treatment of a nail disease. The use involves placing the artificial nail comprising the one or more reservoirs with the liquid composition on the infected nail or nail bed such that the liquid composition comprising the antimicrobial agent is in direct contact with the nail to be treated and the artificial nail is sealing (in an occlusive manner) the nail or nail bed to be treated.

The present invention will be further detailed in the following examples and figures wherein:

FIG. 1: shows a fungus infected nail (1) on a foot which is treated by application of the artificial nail (2) of present invention. When applied to the nail or nail bed to be treated, the artificial nail fully seals off the infected nail from the environment, following the contours of the natural nail and nail bed. The nail-specific sealing (i.e. occlusion that prevents evaporation of the liquid solution comprising the antimicrobial compound) allows sufficient time for the antimicrobial compound to reach the site of action in sufficient quantities in order to be effective, thus improved transungual delivery of the active compound to the site of infection or disease. Under sealing or occlusion conditions, the nail has a better ability to absorb the liquid composition as well as the solubilized drugs therein, which means that the liquids comprising the solubilized drugs can readily diffuse to the deeper layers of the nail.

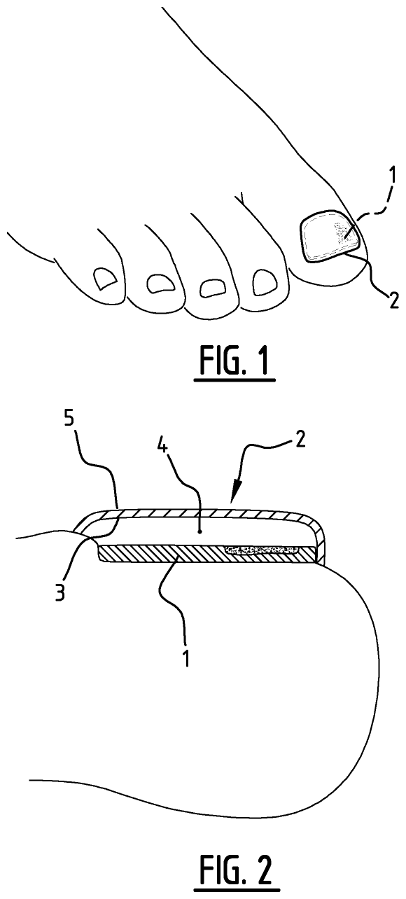

FIG. 2: shows a side view of the artificial nail (2) of present invention comprised of a polymer surface side (5) resembling the keratinous plate of a nail and a ventral side (3) that is to be directed to a nail and/or nail bed to be treated. The ventral side comprises one or more reservoirs (4) for holding a liquid composition comprising an antimicrobial compound for treatment of the infected nail and/or nail bed (1) to be treated. The reservoir of the artificial nail hold a liquid composition comprising an antimicrobial compound for treatment of the infected nail and are on the inner surface of the ventral side facing the surface area to be treated of the nail of nail bed of a finger or toe where the antimicrobial compound needs to be applied to for treatment. The artificial nail of present invention provides a continued administration of the active antimicrobial compound to the site of infection over an extended period of time.

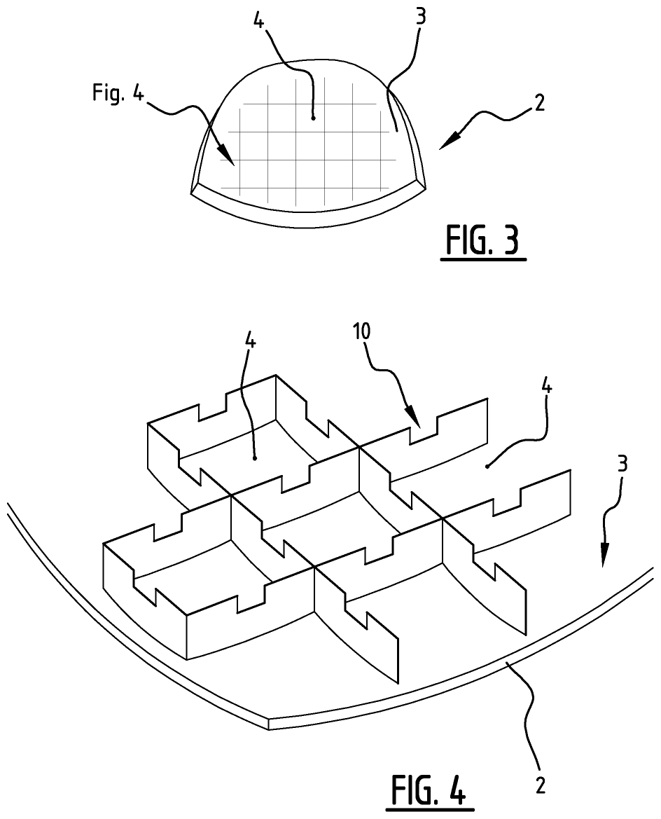

FIG. 3: shows the ventral side (3) of the artificial nail (2) of present invention. The ventral side comprises multiple reservoirs (4) for holding a liquid composition comprising an antimicrobial compound for treatment of the infected nail.

FIG. 4: shows a blow up of the ventral side (3) of the artificial nail (2) comprising the multiple reservoirs (4) for holding a liquid composition comprising an antimicrobial compound for treatment of the infected nail. The reservoirs are comprised of a liquid permeable polymer matrix enabling flow trough of the liquid composition providing a liquid connection between reservoirs, and contact between the liquid composition and infected nail or nail bed to be treated. The permeable polymer matrix may also comprise small apertures (10) to further improve the distribution of the liquid composition in the artificial nail within the one or more reservoirs (4), enabling flow and direct and continues contact with the infected nail or nail bed during the duration of the treatment when the artificial nail (2) is positioned on the infected nail or nail bed for improved transungual delivery of the active compound to the site of infection or disease and efficacy of the treatment. These apertures (4) may be located anywhere in the liquid polymer matrix of the reservoir (4) providing a liquid connection between reservoirs, however apertures located close to the infected nail or nail bed to be treated are preferred for optimal and continues contact of the liquid with the infected nail or nail bed during the duration of the treatment.

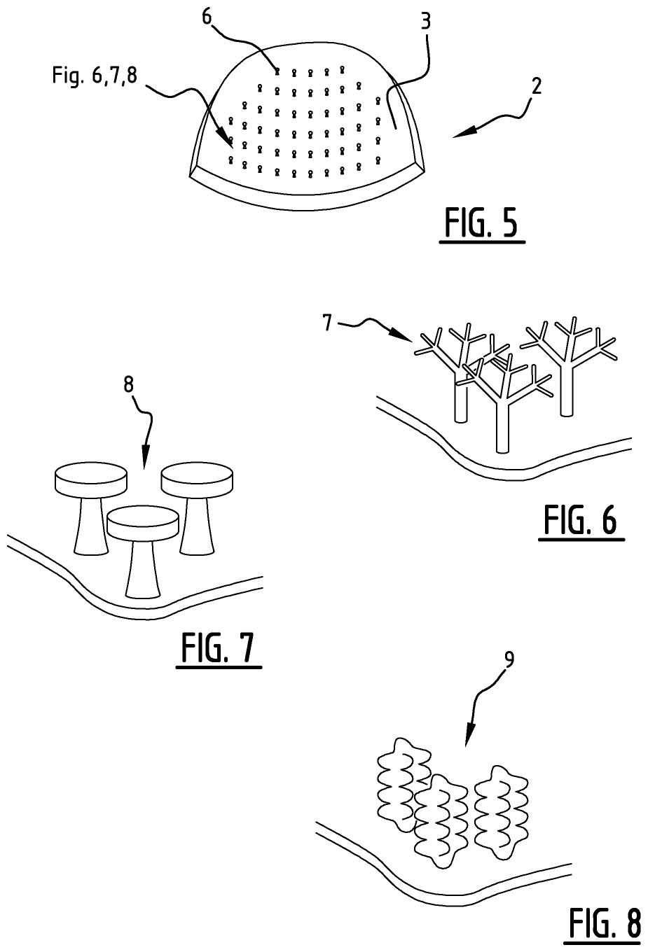

FIG. 5: shows the ventral side (3) of the artificial nail (2) of present invention. The ventral side comprises one or more protrusions (6) that may have a specific 3D shape for providing adhesion of said liquid composition to said ventral side. The specific 3D shape of the protrusions provides an improved adhesive contact area and increase surface tension for the liquid composition from the one or more reservoirs that is held between the infected nail and the ventral side of the artificial nail.

FIG. 6-8: Specific 3D structures, such as tree-like (7), mushrooms (8), or gyroid (9) improve the adhesive contact area and increase surface tension for the liquid composition to the ventral side of the of the artificial nail (2) of present invention, and prevent drip off and spillage of the liquid composition during application of the artificial nail to the site to be treated.

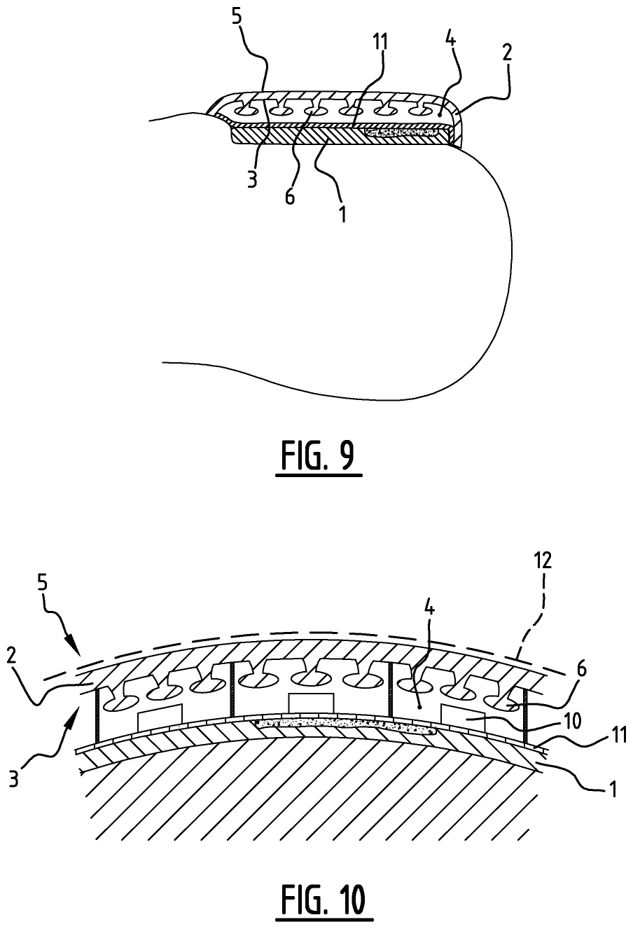

FIG. 9: shows a side view of the artificial nail (2) comprised of a polymer surface side (5) resembling the keratinous plate of a nail and a ventral side (3) that directed to the infected nail. The ventral side comprises a reservoir (4) for holding a liquid composition comprising an antimicrobial compound for treatment of the infected nail and multiple protrusions (6) that provide an improved adhesive contact area for the liquid composition in the reservoir. The reservoir (4) is comprised of a liquid permeable polymer matrix that is comprised of an adhesive polymer material (11) to improve adherence of the artificial nail and to provide a further enclosure of the reservoir (4)

FIG. 10: shows a cross section of the artificial nail (2) of present invention comprised of a surface side (5) resembling the keratinous plate of a nail and a ventral side (3) that is directed to the infected nail (1). The ventral side comprises reservoirs (4) for holding a liquid composition comprising an antimicrobial compound and protrusions (6) for providing adhesion of said liquid composition to the ventral side of the polymer layer of the artificial nail. The reservoirs of the artificial nail (4) are comprised of a liquid permeable polymer matrix that is comprised of an adhesive polymer material (11) layer that is liquid permeable and allows flow trough of the liquid composition and contact between the liquid composition and nail and/or nail bed to be treated, and to improve adherence of the artificial nail and to provide a further enclosure of the reservoir (4). The reservoirs (4) further comprise small apertures (10) to further improve the distribution of the liquid composition within the one or more reservoirs (4), enabling flow and direct and continues contact with the infected nail or nail bed (1) during the duration of the treatment. Furthermore, the artificial nail may be further covered by an adhesive top layer (11), for example Tegaderm.

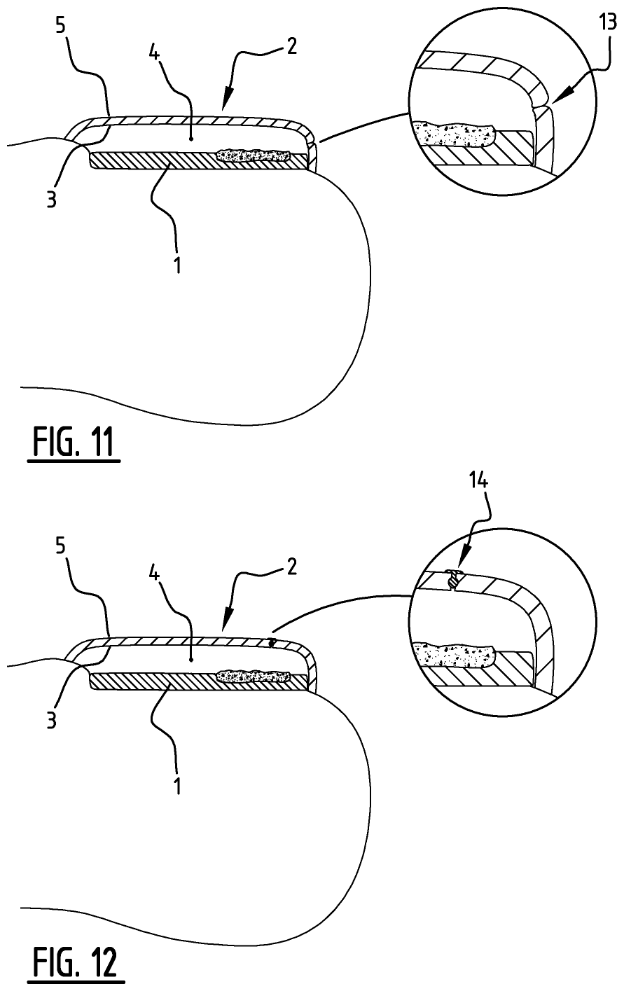

FIG. 11: shows a cross section of the artificial nail (2) of present invention comprised of a surface side (5) resembling the keratinous plate of a nail and a ventral side (3) that is directed to the infected nail (1). The ventral side comprises reservoir (4) for holding a liquid composition comprising an antimicrobial compound. The polymer layer of the artificial nail (2) comprises a valve (14) for adding or (re)filling of the reservoir (4).

FIG. 12: shows a cross section of the artificial nail (2) of present invention comprised of a surface side (5) resembling the keratinous plate of a nail and a ventral side (3) that is directed to the infected nail (1). The ventral side comprises reservoirs (4) for holding a liquid composition comprising an antimicrobial compound. The polymer layer of the artificial nail (2) comprises an aperture in combination with a closing means (14), such as a plug, for adding or (re)filling of the reservoir (4).

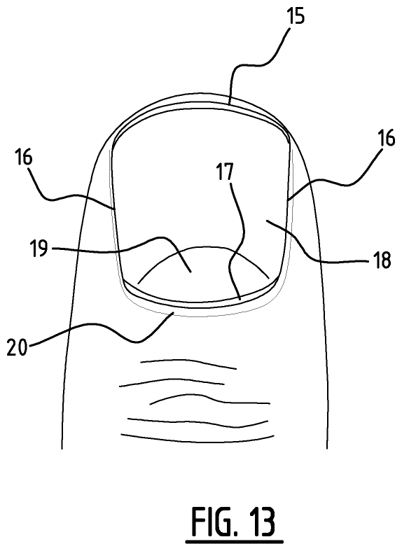

FIG. 13: shows a healthy nail and its anatomy; the distal edge or end of the nail plate (15), the lateral nail folds (paranychium) (16), cuticle (eponychium) (17), nail plate (18), lunula (19), and proximal nail fold (20). The artificial nail is suitable to be applied onto a nail and/or nail bed by attachment to the edge of the cuticle, lateral nail folds and epidermis below the nail plate, preferably at a distal end of the nail plate having a maximum distance to the distal end of the nail plate of at most 1 mm. The distance between the artificial nail to the lateral nail folds and cuticle of said nail and/or nail bed to be treated is at most at most 1 mm.

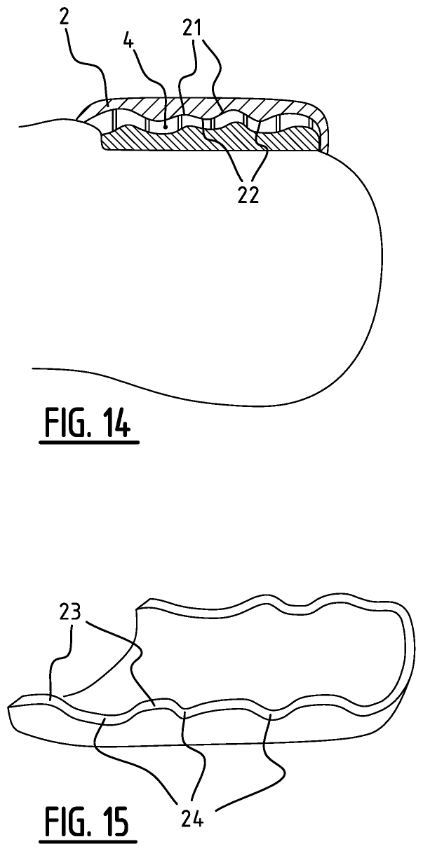

FIG. 14: shows an artificial nail (2) according to an embodiment of present invention, wherein the artificial nail surface at the ventral side (3) is comprised of multiple convex (21) and concave (22) surfaces following the shape of the surface of the nail and/or nail bed to be treated (1). The ventral side comprises reservoirs (4) for holding a liquid composition comprising an antimicrobial compound.

FIG. 15: shows an artificial nail (2) according to an embodiment of present invention, wherein the artificial nail surface at the ventral side (3) is comprised of multiple convex (23) and concave (24) surfaces following the contours of the nail and/or nail bed to be treated (1).

### EXAMPLES

### Example 1—Ability to Maintain High Water Content and Humidity at Site of Infection

For effective treatment of fungal nail disease, it is known that 100% relative humidity (RH) or high water content promotes transungual drug delivery resulting in a more effective and efficient treatment of the affected area of the nail. In this experiment known nail disease treatments; cremes, liquids and lacquers have been tested in comparison to the artificial nail of present invention, where they were applied on a semipermeable membrane (semi-permeable transparent wound dressing films made of polyurethane with acrylic adhesive Tegaderm) that mimicked the nail conditions. Before application of treatment, membranes were weighed on a calibrated scale (Mettler Toledo). Treatments were applied and kept at room temperature for 1 and 24 hours. After 24 hours of exposure membranes were cleaned using wipes (Kimtech). Membranes were weighed again to approximate water content.

As was observed in this experiment that with known treatments such as cremes, liquids or lacquers for topical application, the water content after 1 hour dropped to well below 100%. However, with the artificial nail of present invention the water content remained close to 90% and maintains a high hydration state after even 24 hrs. Due to evaporation and quick removal of liquid formulations, current state of the art does not prefer the use liquid formulations for the treatment of nail disease. However, results show that high water content and hydration state, more specifically using aqueous liquid compositions in combination with the artificial nail of present invention promotes transungual drug delivery to the site of infection or disease and improve treatment.

### Example 2—Nail Penetration Assay

In this experiment the penetration of the antimicrobial compound in a nail and the effect of occlusion and hydration on nail permeation of medicinal antifungals is investigated. A collection of human nail clippings were used that were approved by relevant ethical committees. Nail clippings of at least 8 mm long were donated by healthy volunteers after providing written informed consent. Nail thickness varied from 300 μm to 550 μm. Five different formulations each with a different antifungal (Terbinafine, Ciclopirox, Voriconazole, Chlorhexidine and Efinoconazole were used, obtained from Novartis, Pierre Fabre Dermatologie, Sandoz, Cedium, Valeant, and Ortho Dermatologics), were compared in an occluded and a non-occluded modified Franz Diffusion cell set up.

The nail penetration of the antifungal formulation in occlusion state was tested using a vertical diffusion cell setup or a modified Franz diffusion cell with a small 0.5 mL receptor compartment was used (Permegear.com), comprised of a donor chamber, a mount with a nail or semi permeable membrane, a receptor chamber with stirrer. The nail is mounted between the donor chamber and receptor chamber, wherein the ventral side of the nail is directed to the receptor chamber.

The receptor solution (5 mL) was phosphate buffer saline (pH 7.4) with 0.5% of polysorbate 80 and was stirred. Nail tips were hydrated for 30 minutes in deionized water. Nail tips were placed in a nail adapter which was sandwiched between the donor and receptor compartments of diffusion cells. Each formulation was tested three times, in an occluded and non-occluded setup. For each condition a single dose was applied in the donor compartment. In the occluded condition the donor compartment of the in-line cell setup was covered with non-permeable wrapping to limit evaporation and mimic NAIL-IT conditions. In the non-occluded condition, the solution was administered and the donor compartment was kept open. The assembly was shaken in a horizontal shaker for seven days. Next, receptor samples (0.4 mL) were collected after 24 h and every 24 h thereafter and replaced by fresh medium for a total duration of 7 days. Samples were collected in glass tubes and stored in darkness at <−18° C. until thawed for analysis. The concentration of antifungal in receptor chamber was quantified by HPLC.

Next, the area of the nail exposed to the formulations was cut into small pieces and antifungal was extracted by shaking the fragments with 1 mL of methanol:water (80:20) or other solvent for 7 days. The antifungal concentrations in the extracts were determined by HPLC. The antifungal concentrations in the extracts were determined by LC-MS/MS using the AB Sciex API 3000 triple quadrupole mass spectrometer (Concord, ON, Canada) with an Agilent 1100 series HPLC system (Agilent Technologies, Palo Alto, USA), and a cooled autosampler. The antifungal assays were validated on general validation parameters (accuracy, precision, limits of quantitation, selectivity, matrix effect, recovery and carry-over). Flux (μg·cm⁻²·s⁻¹) was calculated and expressed as micrograms of antifungal per square centimetre of nail per square centimetre. Permeability (cm·s⁻¹) is derived from flux by dividing flux by the starting concentration (μg·cm-3). Table 1 summarizes the results per compound and per test condition, occluded and non-occluded.

TABLE 1

| Antifungal | Measurement | Non-Occluded | Occluded |
|---|---|---|---|
| Terbinafine | Flux (μg · cm⁻² · h⁻¹) | .02 | .05 |
| Ciclopirox | Flux (μg · cm⁻² · h⁻¹) | .4 | 1 |
| Voriconazole | Flux (μg · cm⁻² · h⁻¹) | .30 | .75 |
| Efinaconazole | Flux (μg · cm⁻² · h⁻¹) | .2 | .5 |
| Chlorhexidine | Flux (μg · cm⁻² · h⁻¹) | 1.2 | 6.0 |
| Terbinafine | Recovered nail (%) | 2% | 4% |
| Ciclopirox | Recovered nail (%) | 1% | 2% |
| Voriconazole | Recovered nail (%) | 3% | 15% |
| Efinaconazole | Recovered nail (%) | 2% | 4% |
| Chlorhexidine | Recovered nail (%) | 1% | 5% |

The occluded nails according to present invention provided a significantly improved flux (>50% to 400% increase) and resulting permeability, as well as significant improved total recovery percentage of the treated nail surface, indicating an enhanced therapeutic and cosmetic treatment using the artificial nail of present invention.

### Example 3—In Vivo Case Study

The artificial nail of present invention was tested on a subject with mild to moderate onychomycosis and maximally 3 affected nails. All affected nails will be treated, to avoid the risk of co-infection of the untreated nails during the procedure. The treatment procedure for the infected toenail is described below.

The affected and thickened nail was abraded to normal non-infected proportions and removed as much as possible. Abrasion was done to such an extent that no direct damage was made to the residual nail or nail plate. Subsequently a 3D-scan was made of the affected nail and dimensions (length, width, curvature, outline) were obtained from the affected nail. Based on the nail dimensions, a polymer material (SLA material) in the shape of an artificial nail providing full occlusion of the surface area to be treated was made. Attached to the polymer material were reservoirs of polypropylene having a 3D mushroom structure, wherein the reservoirs were loaded (approx. 200 ul) with antifungal fluid composition, i.e. a formulation containing itraconazole as the active antifungal. The formulation contained 10 mg/ml of itraconazole, in a solution containing propylene glycol, hydroxypropyl-b-cyclodextyrin, hydrochloric acid, sodium hydroxide and water. A polypropylene sponge is placed in the reservoirs to hold the fluid composition in place. The open structure of this material allows direct access of the itraconazole solution to the nail. Occlusive conditions are maintained for the duration of the application.

Before application of the nail of present invention, a picture at t=0 was made of the nail. Immediately thereafter, the nail was fixed by cyanoacrylate gluing of the contours of the nail and nail bed and coverage of the nail by an oversized sheet of Fixiomull (BSN medical). The nail was applied for at least 8 hours for 36 consecutive days and after treatment the nail was removed and the treated nail and area was visually observed and photographed. Then the nail was left untreated for three weeks followed again by visual observation. Furthermore, the above procedure was also done for a daily application cycle that was repeated 36 times (a total of 5 weeks). For each application cycle, the antifungal solution in the reservoirs was replaced with fresh solution.

Results show that no leakage or spillage took place during treatment when applied properly. Visual improvement of the infected nail was observed during the first 30 days, demonstrated by healthy nail outgrowth, afterwards improvement halted, and treatment was halted. Wearing comfort was indicated as “good” by the test subject.

1. An artificial nail for use in the treatment of a nail disease, wherein the artificial nail is in the shape of a nail for sealing of a nail and/or nail bed surface of a nail, wherein the artificial nail comprises:
a polymer layer comprising a surface side resembling the keratinous plate of a nail and a ventral side directed to a nail and/or nail bed to be treated,
wherein said ventral side comprises one or more reservoirs for holding a liquid composition comprising an antimicrobial compound and/or wherein said ventral side comprises one or more protrusions for providing adhesion of said liquid composition to the ventral side of the polymer layer, and
wherein said one or more reservoirs is configured to allow application of said liquid composition comprising an antimicrobial compound to said nail and/or nail bed to be treated.

2. The artificial nail according to claim 1, wherein said one or more reservoirs comprises a liquid permeable polymer matrix for enabling flow through of the liquid composition and contact between the liquid composition and nail and/or nail bed to be treated, wherein the liquid permeable polymer matrix is comprised of one or more materials selected from the group consisting of keratin, polymethylmethacrylates, polyamide, polyester, polyether imide, polyethersulfone, polyethylenes, polyglycolide, polyglycolide, polyisocyanates, polylactide, polypropylene, polysiloxanes, polystyrene, polyvinylchloride, polytetra-fluoroethylens, polyvinyl butyral, polyurethane, cellulose acetate, chitosan, collagen, modified polyacrylonitrile.

3. The artificial nail according to claim 1, wherein said one or more protrusions have a 3D shape selected from the group consisting of mushroom, tree, gyroid, cubical, cone, cylinder, pyramid, and sphere.

4. The artificial nail according to claim 1, wherein the polymer layer is comprised of one or more materials selected from the group consisting of keratin, poly methylmethacrylate, polyacrylate, polycarbonate, polyether, polyethersulfone, polyethylene terephthalate, polyisocyanate, polypropylene, polysiloxane, polystyrene, polysulfone, polyvinylchloride, polyvinyl butyral, polyurethane, cellulose acetate, chitosan, epoxie, and combinations thereof.

5. The artificial nail according to claim 1, wherein said one or more reservoirs comprises a liquid permeable polymer matrix that is further comprises an adhesive polymer material selected from the group consisting of resin, rosin, TSF resin, polyepoxides, copal, latex, gum resin, styrax liquid, methyl salicylate, methacrylates, acrylates, diacrylates, triacrylates, sobomyl acrylate, dimethylacrylamid, ethyl cyanoacrylate, methacrylic acid, methyl methacrylate, N-butyl methacrylate, iso-butyl methacrylate, ethyl methacrylate, 2-ethylhexyl methacrylate, N,N-dimethyl-p-toluidine, 2-(2-hydroxy-5-methylphenyl) benzotriazole, 2-hydroxy-4-(ocyloxy) benzophenone, (2H-benzotriazol-2-Yl)-4,6-ditertpentylphenol, polyfunctional amines, isophorone diamine, and combinations thereof.

6. The artificial nail according to claim 1, wherein the one or more reservoirs comprise a volume of 5 to 250 pl.

7. The artificial nail according to claim 1, wherein the one or more reservoirs are at least two reservoirs.

8. The artificial nail according to claim 1, wherein the artificial nail comprises a rigid structure of non-uniform thickness, wherein the dorsal side of said nail has a smooth surface whereas the ventral side will follow the nail and/or nail bed to be treated.

9. The artificial nail according to claim 1, wherein the artificial nail surface at the ventral side is comprised of multiple convex and concave surfaces following the shape of the surface of the nail and/or nail bed to be treated and/or following the contours of the nail and/or nail bed to be treated.

10. The artificial nail according to claim 1, wherein the polymer layer is comprised of at least one UV-curable polymer.

11. The artificial nail according to claim 1, wherein the polymer layer of the artificial nail comprises at least one valve or at least one aperture in combination with a closing means, such as a plug, for (re)filling of the one or more reservoirs for holding a liquid composition comprising an antimicrobial compound.

12. A method for treating a nail disease using an artificial nail, comprising:
a) providing the artificial nail according to claim 1,
b) preparing the artificial nail by adding a liquid composition comprising an antimicrobial compound to the one or more reservoirs, and
c) applying the prepared artificial nail onto a nail and/or nail bed to be treated thereby sealing off the nail and/or its nail bed from the environment.

13. The method for treating a nail disease using an artificial nail according to claim 12, wherein applying the prepared artificial nail comprises that the one or more reservoirs are in direct contact with the nail and/or its nail bed to be treated.

14. The method for treating a nail disease using an artificial nail according to claim 12, wherein the liquid composition comprising an antimicrobial compound is a solution comprising water and/or alcohol, propylene glycol, hydroxypropyl-b-cyclodextyrin, hydrochloric acid, sodium hydroxide and an antimicrobial compound; and/or
wherein the liquid composition further comprises one or more compounds comprising a sulfhydryl (—SH) group selected from the group consisting of acetylcysteine, cysteine, mercaptoethanol, and thioglycolic acid.

15. (canceled)

16. The method for treating a nail disease using an artificial nail according to claim 12, wherein abrasion of said nail and/or nail bed to be treated is performed before application of the prepared artificial nail onto to a nail and/or nail bed to be treated.

17. The method for treating a nail disease using an artificial nail according to claim 12, wherein the antimicrobial compound is one or more selected from the group consisting of antimycotic agent, itraconazole, tavaborole, efinaconazole, terbinafine, miconazole, clotrimazole, bifonazole, butoconazole, econazole, fenticonazole, ketoconazole, oxiconazole, sulconazole, voriconazole, albaconazole, fluconazole, ravuconazole, amorolfine, butenafine, chlorhexidine naftifine, andilafiingin, caspofungin, micafiingin, benzoic acid, ciclopirox, tolnaftate, undecylenic acid, crystal violet, methylene blue.

18. The method for treating a nail disease using an artificial nail according to claim 12, wherein the nail disease is one or more selected from the group consisting of onychomycosis, pseudomonas infection, psoriasis of the nail, and paronychia.

19. The method for treating a nail disease using an artificial nail according to claim 12, wherein the prepared artificial nail is kept onto said nail and/or nail bed to be treated for a 20 period of at least 1 day.

20. The method for treating a nail disease using an artificial nail according to claim 12, wherein method is a cosmetic method of treating a nail disease.

21. The method for treating a nail disease using an artificial nail according to claim 12, wherein the artificial nail is applied onto a nail and/or nail bed by attachment to the edge of the cuticle, lateral nail folds and epidermis below the nail plate; and/or wherein the distance between the artificial nail to the lateral nail folds and cuticle of said nail and/or nail bed to be treated is at most 1 mm.

22-23. (canceled)
